(19) United States
(12) Patent Application Publication
(10) Pub. No.: US 2010/0234680 A1
(43) Pub. Date: Sep. 16, 2010

(54) METHOD OF TREATMENT OF PROLAPSES

(76) Inventor: GEORGES EGLIN, BEZIERS (FR)

Correspondence Address: CANTOR COLBURN, LLP, 20 Church Street, 22nd Floor, Hartford, CT, 06103, US

(21) Appl. No.: 12/401,374

(22) Filed: Mar. 10, 2009

(51) Int. Cl. A61F 2/00

(52) U.S. Cl. 600/37

(57) ABSTRACT

The present invention relates to a method of treatment of a urogenital prolapse, said method comprising the following steps:
a) an incision is made in the vaginal area in order to reach the uterosacral ligament,
b) an intraligamentous dissection of the uterosacral ligament is performed,
c) a reinforcement of the uterosacral ligament is introduced into the dissected intraligamentous part.

The present invention relates to a method of surgical treatment of pelvic floor disorders principally in women, in particular genital prolapses, using the anatomical approach within the uterosacral ligament.

Prolapses are phenomena affecting an organ or part of an organ that has sunk downwards, for example in the region of the vaginal wall. Prolapsed organs may be the uterus, the bladder, the rectum and the small intestine. The present invention concerns in particular the treatment of prolapses of the female genital tract in the region of the uterus.

Treatment of urogenital prolapses requires the correction of anatomical defects in the anterior, central and posterior subperitoneal perineopelvic regions. These defects can be global or elective in one or other of three sectors: below the bladder (anterior), uterine (central) or prerectal (posterior).

Surgical correction can be performed using a high approach or low approach.

Using a high approach or abdominal approach, the central or uterine sector is usually corrected by promontofixation. This technique involves a surgical tape, generally made of biocompatible synthetic material, being placed between the sacral promontory and the cervical region by laparotomy and, increasingly today, by celioscopy. Supplementary fixation of the anterior and posterior sectors can be performed using the same approach.

However, the anatomical results of promonto-fixation remain inadequate for the anterior and posterior sectors. Moreover, the use of this technique has the following disadvantages: the axis of correction resulting from this fixation technique is oriented towards sacral vertebra S1, whereas the anatomical axis of the upper part of the vagina is oriented towards sacral vertebra S3. Thus, the natural anatomical axis is not respected. Moreover, the intervention requires opening of the peritoneum, that is to say of the abdomen, and poses considerable risks of infection or surgical risks (damage to the digestive tract, urethra or bladder).

Using a low approach, the central or uterine sector is usually corrected by sacrospinous fixation using the Richter technique. This technique involves fixing the vaginal fundus or uterine neck directly or indirectly to the sacrospinous ligament. This manoeuvre requires a vaginal incision that is sufficiently large to permit dissection of the sacrospinous ligament. Here too, the axis of correction is different from the natural anatomical axis. Moreover, this technique is less surgically reproducible than promontofixation.

One aspect of the present invention is to provide a novel method of treatment of pelvic floor disorders, principally in women, using a novel anatomical approach, namely an intraligamentous approach allowing the presacral space to be reached and permitting insertion of a synthetic or biological reinforcement into the uterosacral ligament as far as its point of insertion on the sacrum, then in the presacral space; this tape, after it has been passed through the ligament, may optionally be fixed in the area of the uterine points of insertion of the uterosacral ligaments.

Another aspect of the present invention is to provide a technique that permits correction of prolapses in a strictly anatomical manner by reinforcing and shortening the uterosacral ligaments, which are one of the key elements of the pelvic floor.

The present invention concerns a method of treatment of a urogenital prolapse, said method comprising the following steps:
a) an incision is made in the vaginal area in order to reach the uterosacral ligament,
b) an intraligamentous dissection of the uterosacral ligament is performed,
c) a biological or synthetic reinforcement of said uterosacral ligament is introduced into the dissected intraligamentous part.

As a biological reinforcement of the uterosacral ligament, it is possible to use human or animal tissue grafts, for example tissue from pig skin, or engineered tissues obtained by culturing stem cells. As a synthetic reinforcement of the uterosacral ligament, it is possible to use surgical tapes. The surgical tapes suitable for the method according to the invention can be any of the tapes customarily used in the treatment of prolapse or of urinary incontinence. Such a tape can, for example, be a lattice or a knit made from biocompatible synthetic filaments, for example of polypropylene.

In one embodiment of the method according to the invention, step b) is preceded by a step b0) involving extraligamentous dissection of said uterosacral ligament.

In one embodiment of the method according to the invention, said intraligamentous dissection is started from an entry point situated in the median and pararectal part of said uterosacral ligament.

In one embodiment of the method according to the invention, said intraligamentous dissection is performed using a dilator which is introduced into the uterosacral ligament and is pushed forward within said ligament, in a longitudinal direction of said ligament, while keeping said ligament tensioned. For example, said dilator is advanced within said ligament until it comes into contact with the presacral osseous plane.

The path of said dilator is verified by intrarectal digital palpation.

In one embodiment of the method according to the invention, an end of said reinforcement is introduced into the dissected part as far as the presacral osseous plane. Said end of said reinforcement can be left free. In such a case, the reinforcement can fasten naturally to the surrounding tissues, simply as a result of the friction between the reinforcement and the surrounding organic tissues, or, for example when the reinforcement is a tape, because the tape has rough edges or has side filaments that are left free, for example on account of a specific knitting scheme.

Alternatively, said end of said reinforcement is fixed to said presacral osseous plane with the aid of an anchoring device. Such an anchoring device can be chosen from among sutures, staples, clips, adhesives or any other biocompatible means of fixation.

In one embodiment of the method according to the invention, the part of said ligament reinforced by said reinforcement is sectioned before said point of entry and is fixed in the area of the torus uterinus. The reinforced part of said ligament may be fixed to the torus uterinus by means of a non-absorbable suture point.

In one embodiment of the method according to the invention, an endoscope is introduced into the dissected intraligamentous part after withdrawal of said dilator. Subperitoneal insufflation can then be performed in order to view the presacral region.

In one embodiment of the method according to the invention, an optical instrument is introduced into the dissected intraligamentous part after withdrawal of the dilator.

In one embodiment of the method according to the invention, the uterosacral ligament is located during step b0) by exerting firm traction on the neck of the uterus in the longitudinal axis of said ligament.

The present invention will now be described in detail with reference to the attached figures, in which:

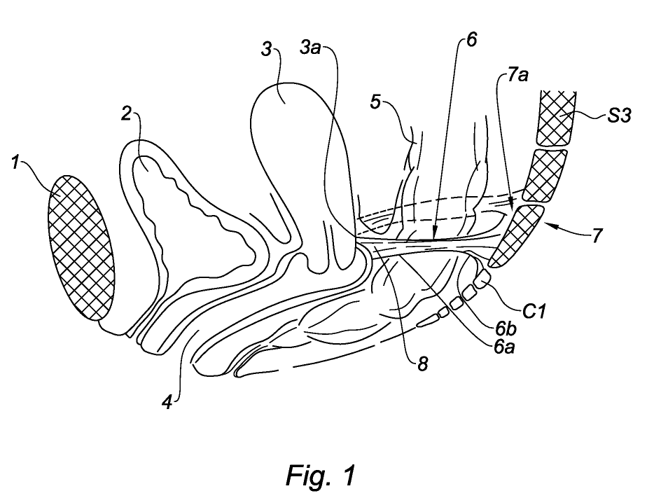

FIG. 1 is a schematic view of a sagittal section of the subperitoneal pelvic space in women,

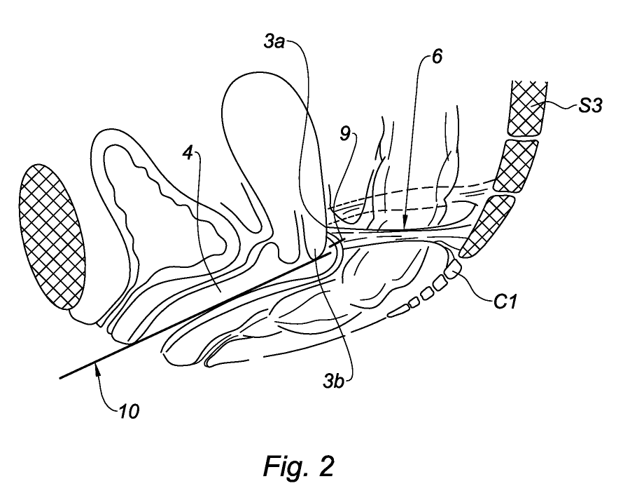

FIG. 2 is a schematic view of the pelvic space from FIG. 1 showing the incision made in step a) of the method according to the invention,

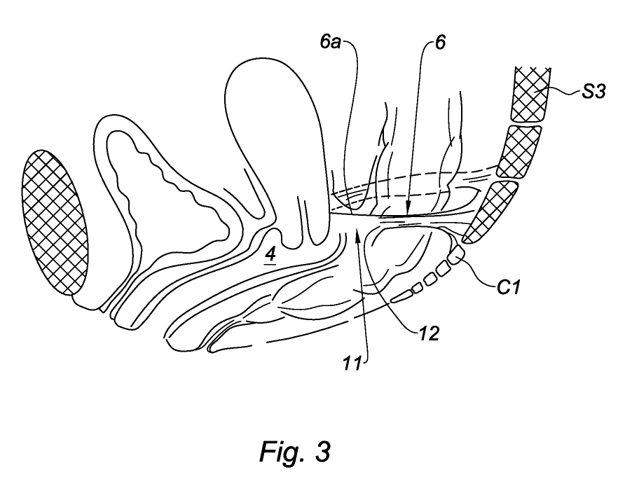

FIG. 3 is a schematic view of the pelvic space from FIG. 2 after the extraligamentous dissection according to step b0) of the method according to the invention,

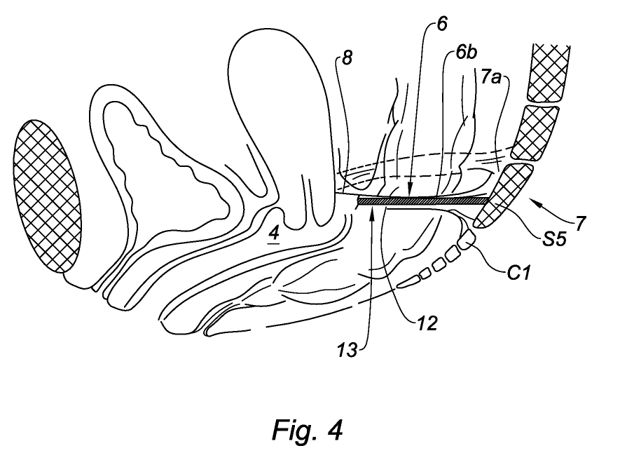

FIG. 4 is a schematic view of the pelvic space from FIG. 3 after the intraligamentous dissection according to step b) of the method according to the invention,

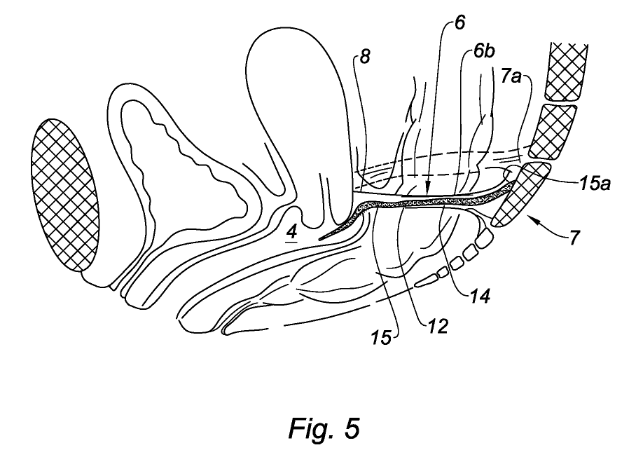

FIG. 5 a schematic view of the pelvic space from FIG. 4 showing the introduction of a tape according to step c) of the method according to the invention,

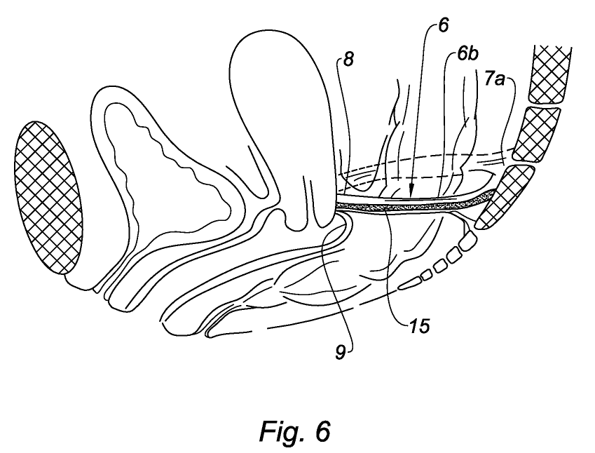

FIG. 6 is a schematic view of the pelvic space from FIG. 4 once the part of the ligament reinforced by the tape has been fixed in the area of the torus uterinus and the vaginal incision has been closed.

Referring to FIG. 1, the subperitoneal pelvic space is shown schematically in this figure, with the pubis 1, the bladder 2, the uterus 3, the rectum 5 and the osseous plane of the sacrum 7. The figure also shows the left uterosacral ligament 6, which connects the uterus 3 to the presacral osseous plane 7. The uterosacral ligaments are twinned and symmetrical connecting structures (left and right) that extend from the isthmus 3a of the uterus to the sacrococcygeal bone structures 7a; each uterosacral ligament 6 comprises a ventral end 6a, which is continuous with the isthmus structures 3a so as to form the torus uterinus 8, and a widened dorsal end 6b, which describes a sacrococcygeal insertion line running from the third sacral vertebra S3 to the first coccygeal vertebra C1; in this area the ligament insertion points are continuous with the presacral fascia.

The uterosacral ligament takes the form of a lamina comprising an inner face, which adheres intimately to the peritoneum in its ventral part, and then, more loosely, to the lateral and subperitoneal face of the rectum. Its outer face is directly in contact with the cephalic part of the pararectal space.

In the event of a prolapse, the uterosacral ligaments are no longer able to serve their purpose as a means of maintaining the equilibrium of the pelvic floor, as a consequence of which some organs, such as the uterus, are no longer adequately supported and suffer prolapse.

The surgical technique in the method of treatment of a urogenital prolapse according to the invention, using the uterosacral intraligamentous approach, will now be described with reference to FIGS. 2-5.

Referring to FIG. 2, a high and posterior incision 9 is made in the area of the vagina 4, or a colpotomy, with the aid of a bistoury 10 or scissors, in the longitudinal or transverse direction, for example for about 4 cm.

Such an incision 9 affords sufficient clearance to allow the surgeon to grip the end of the uterosacral ligaments 6 in the area of their point of insertion on the isthmus 3a of the uterus, for example with the aid of Allis forceps; the uterosacral ligament 6 is made easier to locate by exerting a firm traction on the neck 3b of the uterus in the longitudinal axis of the ligament 6; this traction can be effected using Pozzi forceps, for example.

Once the uterosacral ligament 6 has been reached and its end gripped, the surgeon can begin the extraligamentous dissection of the ligament 6. The extraligamentous dissection of the uterosacral ligament 6 starts, for example, at its lower and outer margin below the peritoneum and can be continued for approximately 6 cm or more, depending on the extent of the prolapse: this distance corresponds approximately to the ventral portion of the ligament bordering Douglas's pouch. This dissection can be performed using Mayo scissors, which causes less hemorrhaging. In one embodiment of the method according to the invention, a Breisky valve may be fitted. Such a valve allows the pararectal tissues to be pushed aside in order to expose the first part of the uterosacral ligament while remaining outside said uterosacral ligament. FIG. 3 shows the anatomical structure from FIG. 2 once the extraligamentous dissection of the uterosacral ligament 6 has been performed: the ventral part 6a of the uterosacral ligament 6 has been dissected and exposed at the location designated by 11 in the figure.

The surgeon can then start the intraligamentous dissection by penetrating the uterosacral ligament 6 from an entry point 12 situated in the median and pararectal part thereof, for example approximately 4 cm from its sacral point of insertion 7a. To do this, it is preferable to keep the uterosacral ligament 6 tensioned and to make a very small incision there, for example with the tip of a bistoury: through this orifice the surgeon introduces a dilator 13, for example of the Hegar No. 5 type which, as it moves through the ligament 6, guided by the tensioned fibres, comes into contact with the anterior face of the first coccygeal vertebra C1, then of the fifth sacral vertebra S5, as is shown in FIG. 4, in which only the distal part of the dilator 13 is depicted. During this step, the path of the dilator 13 can be monitored by intrarectal digital palpation. This manoeuvre in particular avoids any risk of damage to the rectum.

Referring to FIG. 5, the intraligamentous dissection allows a tunnel or a channel 14 to be created within the dorsal part 6b of the uterosacral ligament 6. This channel 14 of the distal portion 6b of the uterosacral ligament 6 will permit introduction therein of a uterosacral ligament reinforcement, in the form of an intraligamentous reinforcement tape 15 in the example shown. Such a tape can be any tape customarily used in the treatment of prolapses or of urinary incontinence. Such a tape can, for example, be a lattice or a knit made from biocompatible synthetic filaments, for example of polypropylene.

In an embodiment not shown, the tape could be replaced by a biological reinforcement, such as a human or animal tissue graft, for example tissue from pig skin, or engineered tissues obtained by culturing stem cells.

As is shown in FIG. 5, the end 15a of a synthetic tape 15, for example measuring about 10 mm in width and 100 mm in length, is introduced into the dissected dorsal part 6b of the uterosacral ligament 6, for example with the aid of the previously used dilator 13 or another suitable ancillary means; the end 15a of the tape 15 is pushed into contact with the presacral osseous plane 7.

The end 15a of the tape 15 can be fixed to the presacral osseous plane 7 using an anchoring device, for example a suture or staple. Alternatively, said end 15a can be left free, the tape fastening naturally in the surrounding tissues. It should be noted that in a second stage, some time after implantation, the new collagen induced by the tape 15 by cell recolonization will contribute naturally to the presacral fixation of the tape 15 and to the strengthening of the uterosacral ligament 6.

The tape 15 is then cut at its opposite end to the length necessary to allow it to extend by a distance corresponding to that of the dorsal part 6b of the uterosacral ligament 6. The dorsal portion 6b of the ligament, reinforced by the tape 15, is sectioned in front of the point of penetration or point of insertion 12 and is then re-implanted in the area of the torus uterinus 8, as is shown in FIG. 6, for example using a nonabsorbable suture. The vaginal incision 9 is then closed.

In cases of previous hysterectomy, it is possible to perform an intraligamentous approach of the uterosacral ligament; after a high longitudinal colpotomy in the posterior face of the vagina, tensioning of the vaginal dome permits identification of the ligament structure; this is made easier by the intrarectal digital palpation. The rest of the intervention can be carried out as described above. The uterosacral ligament with the intraligamentous tape is then fixed to the lateral part of the vaginal dome.

In one embodiment of the method according to the invention, the intraligamentous approach to the presacral space can be supplemented by intraligamentous endoscopy of the presacral region.

In such a case, the manoeuvre is performed in the same way until the formation of the tunnel in the dorsal part 6b of the uterosacral ligament 6, which formation is carried out with the aid of a dilator 13 and then with an endoscope: an endoscope of 5 mm or more can be used. When the end of the endoscope is positioned in the presacral region, subperitoneal insufflation is performed which permits a direct view of the presacral region with, laterally, the presacral sciatic roots. It is also possible to use an intraligamentous route for the passage of an optical instrument and the contralateral route for the passage of an operating instrument.

The method according to the invention is particularly easy to perform and particularly quick and effective. The method according to the invention is minimally invasive and reduces the risks to the vessels and nerves: it permits simple and close monitoring of the path of the dilator. The method according to the invention permits a considerable reduction in the operating time compared to the methods with high or low approach according to the prior art. It also permits a reduction in post-operative pain and therefore in the period of hospitalization of the patient. The method according to the invention does not require the development of new prostheses, since any surgical tape suitable, for example, for supporting the urethra can be used, for example, in the method according to the invention.

1. Method of treatment of a urogenital prolapse, said method comprising the following steps:
a) an incision is made in the vaginal area in order to reach the uterosacral ligament,
b) an intraligamentous dissection of the uterosacral ligament is performed,
c) a biological or synthetic reinforcement of said uterosacral ligament is introduced into the dissected intraligamentous part.

2. Method according to claim 1, in which step b) is preceded by a step b0) involving extraligamentous dissection of said uterosacral ligament.

3. Method of treatment according to claim 1 or 2, in which said intraligamentous dissection is started from an entry point situated in the median and pararectal part of said uterosacral ligament.

4. Method of treatment according to any one of claims 1 to 3, in which said intraligamentous dissection is performed using a dilator which is introduced into the uterosacral ligament and is pushed forward within said ligament, in a longitudinal direction of said ligament, while keeping said ligament tensioned.

5. Method of treatment according to claim 41 in which said dilator is advanced within said ligament until it comes into contact with the presacral osseous plane.

6. Method of treatment according to claim 4 or 5, in which the path of said dilator is verified by intrarectal digital palpation.

7. Method of treatment according to claim 5 or 6, in which an end of said reinforcement is introduced into said dissected part as far as the presacral osseous plane.

8. Method of treatment according to claim 7, in which said end of said reinforcement is left free.

9. Method of treatment according to claim 7, in which said end of said reinforcement is fixed to said presacral osseous plane with the aid of an anchoring device.

10. Method of treatment according to any one of claims 3 to 9, in which the part of said ligament reinforced by said reinforcement is sectioned before said point of entry and is fixed in the area of the torus uterinus.

11. Method of treatment according to claim 10, in which the reinforced part of said ligament is fixed to the torus uterinus by means of a non-absorbable suture point.

12. Method of treatment according to claim 4 or 5, in which an endoscope is introduced into the dissected intraligamentous part after withdrawal of said dilator.

13. Method of treatment according to claim 12, in which subperitoneal insufflation is performed in order to view the presacral region.

14. Method of treatment according to claim 4 or 5, in which an optical instrument is introduced into the dissected intraligamentous part after withdrawal of the dilator.

15. Method of treatment according to any one of claims 2 to 14, in which the uterosacral ligament is located during step b0) by exerting firm traction on the neck of the uterus in the longitudinal axis of said ligament.

16. Method of treatment according to any one of claims 1 to 15, in which said reinforcement is synthetic and is a surgical tape.

17. Method of treatment according to any one of claims 1 to 15, in which said reinforcement is biological and is chosen from among human or animal tissue grafts, for example tissue from pig skin, or engineered tissues obtained by culturing stem cells.
